SEQUENCE LISTING (7 sequences)

SEQ ID NO: 1 - DNA, 474 bp, Homo sapien
datggaggagatgatggcaatgctgccattgctctgtttcagcatgggcaaggctgctgtgctcaggaccacatagctgaggaagttgacctccatgcttcttcgcacagagtggatatcatcgtggaagagagacatagtggtctgtgtgatgtgattgagaatgagcatgtccagtccacccaagagctttcctgcctcaacaacaaatcgctctgcaaaagccatgtcttccatagtgccggcaatgtagtgagcagaggctgctccgagttcaaggcagcgagacaccaccttctggagcccttcctccgaccttgcagtcaataccacatgggctcccatttttgacagatgatatgccatttctcttccgatccctttgctggccccagtgacaattcactttcttcccctggagcatttctggtctgaactcttcatttgtagaatagtagtaacccaggaaagggc

SEQ ID NO: 2 - DNA, 24 bp, Artificial Sequence
  synthetic
dtgcctgggttactactattctac

SEQ ID NO: 3 - DNA, 21 bp, Artificial Sequence
  synthetic
datggaggagatgatggcaat

SEQ ID NO: 4 - DNA, 41 bp, Artificial Sequence
  synthetic
dtaatacgactcactataggatggaggagatgatggcaatg

SEQ ID NO: 5 - DNA, 22 bp, Artificial Sequence
  synthetic
dgccctttgcctgggttactac

SEQ ID NO: 6 - DNA, 40 bp, Artificial Sequence
  synthetic
dtaatacgactcactatagggccctttgcctgggttacta

SEQ ID NO: 7 - DNA, 22 bp, Artificial Sequence
  synthetic
datggaggagatgatggcaatg

(19) United States
(12) Patent Application Publication
(10) Pub. No.: US 2005/0208658 A1
(43) Pub. Date: Sep. 22, 2005

(54) RNA interference mediated inhibition of 11beta hydroxysteriod dehydrogenase-1 (11beta HSD-1) gene expression

(75) Inventor: Thomas W. Castonguay, Columbia, MD (US)

Correspondence Address: ELMORE,CRAIG & VANSTONE, P.C., 209 MAIN STREET, N. CHELMSFORD, MA, 01863, US

(73) Assignee: The University of Maryland, Riverdale, MD (US)

(21) Appl. No.: 10/993,208

(22) Filed: Nov. 19, 2004

Related U.S. Application Data
(60) Provisional application No. 60/624,399, filed on Nov. 2, 2004.
(60) Provisional application No. 60/524,115, filed on Nov. 21, 2003.

(51) Int. Cl. A61K 48/00; C12N 15/85

(52) U.S. Cl. 435/455; 514/44

(57) ABSTRACT

The present invention relates to compositions comprising double stranded RNA capable of inhibiting the expression of the gene encoding 11β HSD-1, and methods of using the compositions in therapeutic, prophylactic, and research methods.

### RELATED APPLICATIONS

This application claims the benefit of U.S. Provisional Application No. 60/624,399, filed on Nov. 2, 2004 and U.S. Provisional Application No. 60/524,115, filed on Nov. 21, 2003. The entire teachings of the above applications are incorporated herein by reference.

### FIELD OF THE INVENTION

The present invention relates to compositions comprising double stranded RNA capable of inhibiting the expression of the gene encoding 11β HSD-1, and methods of using the compositions in therapeutic, prophylactic, and research methods.

### BACKGROUND OF THE INVENTION

Over the past 20 years, an ever-increasing number of mechanisms that influence food intake, caloric intake and body weight have been identified. Although these mechanisms interact at various levels (in the brain, in the gut, in the liver, etc.), one common finding is that the function of each relies upon adrenal glucocorticoids for their normal function in mammals. Until recently, glucocorticoids were considered to be permissive; that is, their presence at low levels in circulation was necessary for the function of many regulatory factors. Hence, a large body literature documented the necessity of minimal levels of circulating glucocorticoids for obesity to be manifest.

Within the past several years there is accumulating evidence that the role of increased glucocorticoid levels in promoting and maintaining obesity is not limited to maintaining elevated titers of the hormones in circulation. Rather, increases in local tissue concentrations have been implicated in mediating enhanced lipid accumulation in adipose tissue and enhanced glucocorticoid-stimulated hepatic activity (especially gluconeogenesis). The enzyme, 11β-hydroxysteroid dehydrogenase (11β-HSD-1), is expressed in adipose tissue, liver and brain. It can function as either a dehydrogenase, converting active glucocorticoid (cortisol in humans, corticosterone (CORT) in rodents) to its inert metabolite (cortisone in humans and 11-dehydrocorticosterone in rodents), or a reductase, converting the inert metabolite back into the active hormone, depending upon local concentrations of steroids and metabolites. Overexpression of the enzyme in genetically engineered mice results in truncal obesity whereas 11β HSD-1 null mice are resistant to obesity. This is believed to be due in part to attenuated hepatic gluconeogenesis (a glucocorticoid-dependent pathway). Obese humans and genetically obese rodents have elevated adipose tissue 11β HSD-1 activity.

Glycyrrhetinic acid does inhibit 11β HSD activity, but without isoform (there are two isoenzymes 11β HSD-1 and 11β HSD-2) specificity (Horigome et al. (1999) Am J. Physiol, 277: E624-E630). Much of what is known about 11β HSD-1 therefore comes from studies of knockout mice or mice that have been genetically engineered to overexpress the enzyme. Masuzaki et a.l (2001) Science, 294: 2166-2170 reported that mice that over express 11β HSD-1 under the control of the enhancer-promoter region of the adipocyte fatty acid-binding protein aP2 gene develop truncal obesity and have impaired glucose tolerance, hyperphagia, and elevated blood lipids and leptin levels. 11β HSD-1 knockout mice have adrenal hyperplasia but attenuated glucocorticoid-induced activation of gluconeogenic enzymes in response to fasting, as well as lower glucose levels in response to stress (Holmes et al.(2001) Mol. Cell Endocrinol., 171: 15-20). One possibility is that these mice compensate for the mutation by increasing adrenal activity so as to maintain homeostasis. Consistent with this, Harris et al. (2001) Endocrinology, 142:114-20, found that these knockout mice have elevated plasma CORT and ACTH levels at the diurnal nadir as well as a prolonged CORT peak. Similar disruptions in glucocorticoid rhythmicity have been reported in human and rodent obesities. Further, these knockout mice have exaggerated ACTH and CORT responses to restraint stress, as well as impaired stress responsivity.

Recent studies (Stulnig et al. (2002) Diabetes, 51, 2426-2433) have shown that 11β-HSD-1 mRNA can be down regulated by Liver X (α and β) receptors (LXRs) agonists in adipocytes and liver in vitro and in vivo. This group concluded that the down regulation of expression and activity of 11β-HSD-1 using LXRs would be expected to ameliorate 11β-HSD-1-mediated effects on metabolism and has the potential to increase insulin sensitivity. In another recent study (Barf et al. (2002) Journal of Medicinal Chemistry, 45: 3813-3815, a small molecule diethylamide derivative was shown to potently inhibit 11β-HSD-1 enzyme thereby attenuating hepatic gluconeogenesis.

Interference RNA (RNAi) is a powerful, relatively-new method to investigate gene function through suppression of gene expression. Long dsRNAs specifically suppress expression of a target gene. However, the RNAi mechanism is currently being investigated but appears to work through smaller dsRNA intermediates. The parent dsRNA is broken down into these smaller fragments (siRNA) in vivo, and this siRNA directs a post-transcriptional breakdown of the targeted mRNA (Zamore et al. (2000) Cell, 101: 25-33, 2000). An unusual feature of this process is that it works non-stoichiometrically and can spread between cells. Intravenous delivery of RNA and DNA was reported in mammals by Liu et al. (1999) Gene Ther., 6:1258-66. Since that time, several other investigators have shown that intravenous injections of long stranded RNA (˜500 nucleotides) can inhibit specific genes without promoting the expected immune response.

The present inventors have developed a novel treatment for obesity and other disease indications responsive to the modulation of 11β HSD-1 using RNAi to suppress native 11β HSD-1 expression.

### SUMMARY OF THE INVENTION

The present invention provides compositions and methods for inhibiting the expression of a gene encoding 11β HSD-1. In accordance with the invention, RNA comprising a double stranded structure (dsRNA) and having a nucleotide sequence which is substantially identical to at least a part of the gene encoding 11β HSD-1 is introduced into a cell in an amount sufficient to cause specific inhibition of the gene expressing 11β HSD-1. Uptake of injected RNA into the liver is both efficient and quick, making this approach ideal for use when attempting to inhibit the expression of 11β HSD-1, an enzyme found in liver, but not in muscle.

As it is believed that glucocorticoids are a necessary component of all obesities and that 11β HSD-1 plays an integral role in regulating intracellular glucocorticoid concentrations, inhibition of the expression of 11β HSD-1 in accordance with the invention has therapeutic uses in the treatment of obesity and related disorders, and any other indications responsive to the inhibition of 11β HSD-1 and in understanding the metabolic role of 11β HSD-1 in mammals.

### DETAILED DESCRIPTION OF THE INVENTION

In a first aspect of the invention, a method is provided for specifically inhibiting the expression of a gene encoding 11β HSD-1 in a cell, the method comprising introducing into a cell an RNA comprising a double stranded structure (dsRNA) having a nucleotide sequence which is substantially identical to at least part of the gene encoding 11β HSD-1. As used herein, inhibition of the gene encoding 11β HSD-1 means an absence or observable decrease in the level of 11β HSD-1 protein and/or mRNA product from the gene. Specificity refers to the ability to inhibit the gene without manifest effects on other genes in the cell.

Inhibition can be confirmed by examination of the outward properties of the cell or organism or by biochemical techniques such as RNA solution hybridization, nuclease protection, Northern Hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). In the context of medical treatment, verification of inhibition of the expression of a target gene may be by observing the absence, or diminution of symptoms of disease in the patient.

The dsRNA comprises a double stranded structure, the sequence of which is substantially identical to at least part of the gene encoding 11β HSD-1. The double-stranded structure may be formed by a single self complementary RNA strand or two separate complementary RNA strands. RNA duplex formation may be initiated either inside or outside the mammalian cell.

The mechanism of RNAi is still being elucidated, however it is believed that before RNAi occurs, longer strands of dsRNA are cleaved by specific nucleotides to generate small fragments of dsRNA also known as small interfering RNAs (siRNA). It is believed that these intermediate siRNAs are guide sequences in complexing with the mRNA that is targeted for destruction. Therefore, the term “dsRNA” as used herein refers to dsRNA that comprises all or a portion of a gene encoding 11β-HSD-1, regardless of whether the dsRNA is substantially identical to all or a portion of the gene encoding 11β-HSD-1 or whether it has been enzymatically cleaved to form siRNA, or whether the siRNA have been prepared chemically and presented to the cell. Preferably siRNA of the invention, whether enzymatically cleaved within the cell or prepared outside of the cell for administration to the cell, comprise double stranded RNA having at least about 18 nucleotides, and more preferably at least about 19, 20, 21, 22, or 23 oligonucleotides in length and comprise at least about 19 base pair duplexes and more preferably about 20, 21, 22, 23 base pair duplexes. There may optionally be an overhang of 2-3 base pairs at each of the 3′ ends.

As used herein the term “substantially identical” is related to the “identity” between the nucleotide sequence of the dsRNA and the gene encoding 11β HSD-1. “Identity” as is known in the art is the relationship between two or more polynucleotide (or polypeptide) sequences as determined by comparing the sequences. In the art, identity also means the degree of sequence relatedness between polynucleotide sequences as determined by the match between strings of such sequences. Identity can readily be calculated by one skilled in the art (see, e.g., Computational Molecular Biology, Lesk, A.M., ed., Oxford University Press, New York, (1988)). In a preferred embodiment of the invention, there is 100% sequence identity between the dsRNA of the invention and the targeted gene or portion thereof. However, dsRNA having 70%, 80% or greater than 90% may be used in accordance with the invention.

In one preferred embodiment, the dsRNA is substantially identical to all or a portion of SEQ ID NO: 1. If the dsRNA comprises a portion of SEQ ID NO: 1, it is preferable that such portion comprise at least about 18 nucleotides, and more preferably at least about 19, 20, 21, 22, or 23 oligonucleotides in length and comprise about 19 base pair duplexes with optional 2-3 nucleotide overhangs at each of the 3′ ends of the dsRNA.

In accordance with the invention, the dsRNA may be produced in vivo or in vitro. Endogenous RNA polymerase of the cell may mediate transcription in vivo, or cloned RNA polymerase can be used for transcription in vivo or in vitro.

dsRNA may be synthesized using recombinant techniques well known in the art (see e.g., Sambrook et al., Molecular Cloning; A Laboratory Manual, 3^{rd} Ed, Cold Spring Harbor Publishers, NY (2001). Thus, bacterial cells can be transformed with an expression vector which comprises the DNA template (sense and antisense templates or self complementary template) from which the dsRNA is to be derived. Alternatively, the cells of the mammal in which inhibition of gene expression is required may be transformed with an expression vector or by other means. Bidirectional transcription of one or more copies of the template may be by endogenous RNA polymerase of the transformed cell or by cloned RNA polymerase (e.g., T3, T7, SP6) coding for the expression vector or a different expression vector. The use and production of an expression construct are known in the art.

Inhibition of gene expression may be targeted by specific transcription in an organ, tissue, or cell type; an environmental condition (e.g., infection, stress, temperature, chemical); and/or engineering transcription at a developmental stage or age, especially when the dsRNA is synthesized in vivo in the mammal. dsRNA may also be delivered to specific tissues or cell types using known gene delivery systems.

dsRNA may be produced by chemical synthesis as is known in the art and as is described in Usman, et al., (1987), J. Am. Chem. Soc., 109:7845; Scaringe, et al., (1990), Nucleic Acids Res., 18:5433; Wincott, et al., (1995), Nucleic Acids Research,. 23:2677-2684; Wincott, et al., (1997), Methods Mol. Bio., 74:59. Chemical synthesis of dsRNA also allows for the chemical modification of dsRNA. Various modifications to nucleic acid dsRNA structure can be made to enhance enzymatic stability, shelf life, half-life in vitro, and other pharmacokinetic advantages. Such modifications include but are not limited to modification of bases (e.g., the use of inosine or tritylated bases), modifications of the dsRNA backbone (e.g., replacing phosphodiester linkages with phosphorothioate and modifications of the ribose unit (e.g., replacing the 2′hydroxyl with 2′OMe). Examples of many of the above-described modifications may be found in Hunziker and Leumann (1995) Nucleic Acid Analogues: Synthesis and Properties, in Modern Synthetic Methods, VCH 331-417, and Mesmaeker et al. (1994) Novel Backbone Replacements for Oligonucleotides in Carbohydrate Modifications in Antisense Research, ACS, 24-39; Eckstein et al., U.S. Pat. No. 5,672,695 and Matulic-Adamic et al., U.S. Pat. No. 6,248,878, all of which are incorporated herein by reference.

In a second aspect, the invention provides a method for treating all forms of obesity and other indications responsive to modulation of the level of 11β-HSD-1 in a patient. The inventors are the first to show that elevated levels of the enzyme and resulting obesity can be “self-induced” as the result of a high fat diet (see, Example 1). This is the first data to show that dietary manipulation can induce an increase in 11β HSD-1 message resulting in obesity. Thus, modulation of 11β-HSD-1 by lowering or eliminating 11β-HSD-1 activity is useful to treat forms of self-induced obesity in the human population that are a result of a high fat diet. Previously, the role of the enzyme in truncal obesity was shown only in animals that were genetically engineered to knock out, or overexpress the enzyme or that carried a genetic mutation that caused their obesity.

Obesity is often associated with other disorders including but not limited to, impaired glucose tolerance, hypertension, coronary thrombosis, stroke, lipid syndromes, hyperglycemia, hypertriglyceridemia, hyperlipidemia, sleep apnea, hiatal hernia, reflux esophagisitis, osteoarthritis, gout, cancers associated with weight gain, gallstones, kidney stones, pulmonary hypertension, infertility, cardiovascular disease, above normal weight, and above normal lipid levels. Selective 1β-HSD-1 inhibitors of the invention as described herein could prevent or ameliorate any of the above conditions. In addition, selective 11β-HSD-1 inhibitors of the invention may be useful in the case where a patient would benefit from reduced platelet adhesiveness, weight loss after pregnancy, lowered lipid levels, lowered uric acid levels, or lowered oxalate levels.

Another indication responsive to modulation of the level of 11β-HSD-1 enzyme is that of age related learning impairments. Selective inhibitors of 11β-HSD-1 as described herein could prevent or ameliorate glucocorticoid associated learning deficits with age. Studies by Yau et al. (2001) PNAS 98, 4716-4721, show that 11β-HSD-1 knockout mice resist glucocorticoid-associated learning impairments with aging, despite elevated plasma levels of active corticoid levels throughout life as result of the knockout. Therefore, the selective inhibitors of the invention may be useful in treating or forestalling related learning impairments.

Additionally, the selective inhibitors of the invention may be used as an adjunct or primary therapy to treat, or prophylactically treat, numerous eating disorders that have the side effect of causing obesity. Such eating disorders include but are not limited to binge eating disorder, compulsive overeating and the like.

Other indications that are responsive to 11β-HSD-1 modulation include type 2 diabetes and metabolic syndrome related disorders such as insulin resistance syndrome and hyperlipidemia. Inhibitors of the invention may be used prophylactically to prevent obesity in patients that may be susceptible to type 2 diabetes. A link has been established between obesity and the onset of type 2 diabetes in humans. See the review by Wyatt, HR. (2003) Prim Care. 2, 267-279. See also, Stulnig et al. (2002) Diabetes, 51, 2426-2433. Likewise, the inhibitors of the invention may be used as the primary therapy to treat insulin resistant disorders such as type 2 diabetes and associated lipid disorders.

In accordance with this second aspect of the invention, a patient is administered an RNA comprising a double stranded structure (dsRNA) having a nucleotide sequence which is substantially identical to at least part of the gene encoding 11β HSD-1. In one embodiment, the dsRNA is substantially identical to all or a portion of SEQ ID NO: 1. If the dsRNA comprises a portion the gene or of SEQ ID NO: 1, it is preferable that such portion comprise at least about 18 nucleotides, and more preferably at least about 19, 20, 21, 22, or 23 oligonucleotides in length and comprise about 19 base pair duplexes with optional 2-3 nucleotide overhangs at each of the 3′ ends of the dsRNA.

dsRNA nucleotides of the invention may be administered alone or in combination with other therapies for the treatment of obesity and other indications that can respond to the level of gene expression of 11β HSD-1. Methods for the delivery of nucleic acid molecules are known and described in the art (see, Akhtar, et al., (1992), Trends Cell Bio., 2:139; Delivery Strategies for Antisense Oligonucleotide Therapeutics, ed. Akhtar (1995), Maurer, et al., (1999) Mol. Membr. Biol, 16:129-140; Hofland and Huang, (1999) Handb. Exp Pharmacol., 137:165-192; and Lee et al, (2000) Acs Symp. Ser., 752:184-192), all of which are incorporated herein by reference. Beigelman et al, U.S. Pat. No. 6,395,713 and Sullivan et al., PCT WO 94/02595, further describe general methods for delivery of nucleic acid molecules. In addition, the nucleotides of the invention can be introduced into a patient by any standard means, with or without stabilizers, buffers, and the like. Delivery systems such as liposomes may also be used. When it is desired to use a liposome delivery mechanism, standard protocols for formation of liposomes can be followed.

In a third aspect of the invention, a pharmaceutical composition is provided comprising a dsRNA having a nucleotide sequence substantially identical to all or a part of the gene encoding 11β-HSD-1 and a pharmaceutically acceptable excipient. In one embodiment, the dsRNA is substantially identical to all or a portion of SEQ ID NO: 1. It is preferable that the dsRNA comprise at least about 18 nucleotides, and more preferably at least about 19, 20, 21, 22, or 23 oligonucleotides in length and comprise about 19 base pair duplexes. Pharmaceutical compositions may include salts and acid addition salts, for example, salts of hydrochloric, hydrobromic, acetic acid, and benzene sulfonic acid.

As used herein the term “pharmaceutical composition” or “formulation” refers to a composition or formulation in a form suitable for administration, e. g., systemic administration, into a cell or patient. Suitable forms, in part, depend upon the use or the route of entry, for example oral, transdermal, or by injection. Such forms should not prevent the composition or formulation from reaching a target cell (i. e., a cell to which the negatively charged nucleic acid is desirable for delivery). For example, pharmaceutical compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the dsRNA molecules of the invention to the targeted tissue. The rate of entry of a drug into the circulation has been shown to be a function of molecular weight or size. The use of a liposome or other drug carrier comprising the compounds of the instant invention can potentially localize the drug, for example, in certain tissue types.

A preferred route of administration is by intravenous injection. Among the acceptable excipients, vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectibles.

A pharmaceutically effective dose is that dose required to prevent, inhibit the occurrence, or treat (alleviate a symptom to some extent, preferably all of the symptoms) of obesity. The pharmaceutically effective dose depends on the composition used, the route of administration, the type of mammal being treated, the physical characteristics of the specific mammal under consideration, concurrent medication, and other factors that those skilled in the medical arts will recognize. Generally, an amount of about 0.1 mg/kg to about 150 mg/kg body weight/day of active ingredients is administered dependent upon potency of the negatively charged polymer (about 0.5 mg to about 7 g per subject per day). The amount of active ingredient that can be combined with the carrier materials to produce a single dosage form varies depending upon the particular mode of administration. Dosage unit forms generally contain from about 1 mg to about 500 mg of an active ingredient. It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

The compositions of the present invention can also be administered to a subject in combination with other therapeutic compounds such as immunosuppressants to increase the overall therapeutic effect. The use of multiple compounds to treat an indication can increase the beneficial effects while reducing the presence of side effects.

In a fourth aspect, the invention also provides an RNA comprising a double stranded structure having a nucleotide sequence which is substantially identical to at least a part of a the gene encoding 11β-HSD-1 for use in medicine, particularly for use in treating obesity and other indications treatable by modulation of the levels of 11β-HSD-1 in a patient.

In a fifth aspect, the invention provides the use of an RNA in the production of an a medicament, for inhibiting the expression the gene encoding 11β HSD-1 in a cell for the treatment of obesity or other indications that are treatable by modulating the expression of 11β HSD-1, the RNA comprising a double stranded structure having a nucleotide sequence which is substantially identical to at least a part of the gene encoding 11β-HSD-1. In one embodiment of this aspect of the invention, the dsRNA is substantially identical to all or a portion of SEQ ID NO: 1. It is preferable that the dsRNA comprise at least about 18 nucleotides, and more preferably at least about 19, 20, 21, 22, or 23 oligonucleotides in length and comprise about 19 base pair duplexes. The medicament will usually be supplied as part of a sterile, pharmaceutical composition which will normally include a pharmaceutically acceptable carrier as is described above.

In a sixth aspect, the invention relates to a method for examining the function and metabolic role of a gene encoding 11β HSD-1 in a cell or organism. In one embodiment, a dsRNA having a nucleotide sequence which is substantially identical to at least a part of the gene encoding 11β HSD-1 is introduced into a cell or organism. The cell or organism is maintained under conditions under which degradation of mRNA of the gene occurs. The phenotype of the cell or organism is then observed and compared to an appropriate control, thereby providing information about the functional role of the gene and the metabolic role of the protein it expresses.

In a seventh aspect, the invention relates to modulating the levels 11β HSD-1 message and activity in cells in a mammal by manipulating dietary intake. In one embodiment, 11B HSD-1 message and activity in tissue can be up regulated by administering a high fat diet to a mammal for a period of time necessary to up regulate 11β-HSD-1 message and/or activity. Methods of dietary manipulation of enzyme message and activity are particularly useful in creating in vivo models for understanding the metabolic role of 11β-HSD-1 in mammals.

The following examples illustrate the role that 11β HSD-1 plays in obesity as well as in methods for preparing the compositions and using the compositions to reduce or inhibit the expression of 11β HSD-1 in a cell. These examples which employ as the agent of the composition, dsRNA molecules that are substantially similar to SEQ ID NO: 1 made by in vitro synthesis merely illustrate embodiments of this invention and do not limit the scope of the invention.

### EXAMPLE 1

### 11β HSD-1 Plays a Role in Obesity that is not Genetically Induced

The Inventors have generated evidence that supports the hypothesis that access to a high fat diet promotes increased 11β HSD-1 message and activity. Two complementary approaches have been used to generate convincing evidence that the high fat diet will in fact induce increased enzyme message. These observations, coupled with recent findings that 11β HSD-1 is elevated in genetically obese rats and in obese humans, suggests that this enzyme plays an important role in several forms of obesity including forms of obesity that are not genetically induced.

Quantitative RT-PCR

Liver samples from 3 rats fed either the high fat diet for 10 weeks, or their standard diet controls were used. Total liver RNA was isolated using Tri-Reagent. DNA contamination was removed, followed by cDNA synthesis. 11β HSD-1 and GAPDH expression levels were then analyzed in separate real time PCR reactions. A melt curve analysis was performed for each reaction to confirm the specificity of the product. The PCR reactions for all samples were carried out in triplicates and the mean of the cycle number at which the fluorescence exceeded the threshold (CT) for GAPDH was subtracted from that of 11β HSD-1 (ΔCT). The percent change in expression between the two treatment groups was then computed as 2^{-ΔΔCT}. ΔΔCT was defined as the difference between ACT of the high fat diet group and ACT of the low fat diet group.

[image]

Sense and antisense riboprobes were developed to estimated 11β HSD-1 message in different tissues. Primers flanking a 473 bp sequence of 11β HSD-1 were purchased, and used in an RT PCR so that a cDNA strand could be isolated and inserted into a plasmid. Maxi Prep kits were used to recover the insert, and antisense and sense linearized templates were made.

Liver RNA samples from 6 rats fed the high fat diet and 6 rats fed the control diet were prepared using Tri-Reagent. Samples were run out on a MOPS gel and transferred onto a methylcellulose membrane. Radiolabeled 11β HSD-1 probe was applied to the blot and allowed to hybridize for 24 h. The blot was then washed and put into a film cassette with BioMax film. The film was developed after 24h of exposure. The blot was then stripped, and labeled with L32. probe (a commonly used “housekeeping gene”) and was then hybridized for an additional 24h. Again, the blot was washed, and put into a film cassette with BioMax film for 24h. Densitometry was then performed, and blot OD volume was calculated for both hybridizations. Finally, a ratio of HSD/L32 was calculated so that even small differences in total RNA could be adjusted for.

Results from these procedures confirm the results of the RT-PCR experiment. Livers from rats fed the high fat diet for 10 weeks had more than twice as much 11β HSD-1 mRNA than did their controls. These data not only confirm and extend our preliminary data above, but further strengthen our initial hypothesis: access to a high fat diet can induce an increase in 11β HSD-1 message. To our knowledge these data are the first to establish that dietary manipulation can affect 11β HSD-1

### EXAMPLE 2

### dsRNA Synthesis

Template Preparation

RNA is extracted from the liver of an adult male Wistar rat using Tri-Reagent according to directions provided by the manufacturer. Briefly, 1 mL of TriReagent is added to a homogenization tube to which 100 mg of frozen liver is added. The sample is homogenized for 30 seconds. The sample is then allowed to come to room temperature for 10 minutes after which 100 μL BCP is added and shaken vigorously for 15 seconds. The sample is then allowed to incubate at room temperature for 5 minutes. It is then centrifuged at 12000 g for 15 minutes at 4° C. The aqueous phase (600 μL) is transferred to RNAse-free 2.0 ml microtubes. One mL of TriReagent is added and vortexed. An additional 100 μL BCP is added and shaken for 15 seconds. The samples are again centrifuged at 12000 g for 15 minutes at 4° C. 800 μl of the aqueous phase is then transferred to a fresh RNAse-free 2.0 mL microtube. One mL of isopropanol is added and mixed by inversion. The sample is stored at room temperature for 10 minutes and then centrifuged at 12000 g for 8 minutes at 4° C. The resulting pellet is rinsed twice with 1 mL 75% ETOH, centrifuging in between at 8000 g for 5 minutes at 4° C. The pellet is then rinsed with 1 mL 95% ETOH, centrifuged at 8000 g for 5 minutes at 4° C. and then allowed to air dry. The pellet is then resuspended in 40 μL DEPC treated water and stored at −80° C.

Primers (SEQ ID NO: 2 and SEQ ID NO 3) flanking a 498 bp sequence (SEQ ID NO 1) of 11β HSD-1 are purchased (University of Cincinnati DNA Core facility), for use in an RT PCR with the liver RNA so that a cDNA strand can be isolated and inserted into a plasmid (pCR4-TOPO cells; Invitrogen, Carlsbad, Calif.). Maxi Prep kits are used to recover the insert from which antisense and sense linearized templates are made.

T7 promoters are added to both the 5′ and 3′ regions of the sense and antisense fragments by PCR. The primers of SEQ ID NOS: 4, 5, 6 and 7 are used to add the T7 promoter. The nucleotide sequences modified by the addition of the T7 promoter region are then used as a template to create dsRNA fragment using an Ambion (Austin, Tex.) Mega script kit according to manufacturer's instructions.

### EXAMPLE 3

### dsRNA Interference in Genetically Obese and Lean Zucker Rats

Intravenous injections of dsRNA described in Example 2 are administered to genetically obese and lean Zucker rats. Using the method of Liu et al. (Gene Ther. 6:1258-66, 1999), up to 3 μg of dsRNA is injected by tail vein into anesthetized genetically obese and lean Zucker rats. Food intake and body weight is monitored daily for several days after injection. Once the appropriate dose of dsRNA has been determined (one that suppresses intake and weight gain for more than 72 hours), the experiment is repeated in another group of rats. Adipose tissue, brains, livers and blood are collected and intracellular corticosterone concentrations and 11β HSD-1 activity are determined in microsomal tissue fractions of each tissue. The samples are initially centrifuged 14,000×g for 20 min; the supernatant is removed and centrifuged at 50,000×g for 2 hours. Aliquots of each sample are used to determine intracellular corticosterone. The HPLC conditions include using a Phenomenex Luna C 18 column (Phenomenex, Torrance, Calif.) with UV detection (Beckman, System Gold 16, Columbia, Md.). The mobile phase consists of 59% 0.1 mol/L phosphoric acid, 30% acetonitrile, and 10% methanol. Plasma corticosterone are measured by RIA kit (ICN Pharmaceuticals). To determine 11β HSD-1 activity the microsomal preparations are incubated at 37° C. in 10 μmol/L corticosterone and 250 μmol/L nicotinamide adenine dinucleotide phosphate (NADP) in Krebs Ringer buffer (118 mmol/L NaCl, 3.8 mmol/L KCl, 1.19 mmol/L KH₂PO₄, 2.54 mmol/L CaCl₂, 1.19 mmol/L MgSO₄, 25 mmol/L NaHCO₃, and 0.2% glucose) for 30 min. Boiling the samples for 10 min terminates the reaction. Aliquots of each are again analyzed for corticosterone by HPLC, and enzyme activity is estimated by adding NAD and excess corticosterone to the sample, incubating it for 30 minutes and then measuring the hormones a second time. Conversion rates of corticosterone (cort) to 11 dehydrocorticosterone (11-dcort) as well as 11-dcort to cort are calculated for each tissue. Additionally, 11β HSD-1 activities are assayed using the method of Wang et al. (Endocrinology 143: 621-626, 2002). Briefly, adipose tissue homogenates are incubated with 100 nm [³ H] CORT in the presence of 3 mm NADP or with 600 nm [³H] 11-dcort in the presence of 3 mm NADPH at 37° C. for 30 min. The reactions are stopped by the addition of methanol and centrifuged, and the steroid present in the supernatant is separated by HPLC using a DuPont Zorbax C8 column. The separated radioactive products (cort and 11-dcort) are detected and quantified by flow scintillation analysis. Protein concentrations are measured by the Bradford assay using a kit (Bio-Rad Lab, Inc., Hercules, Calif.).

The obese rats are expected to dramatically reduce food intake and body weight in response to treatment. By comparison, the lean animals injected with the same dose are unaffected. Adipose tissue and liver samples taken from 11β HSD-1 dsRNA of treated obese and lean rats are expected to have significantly reduced intracellular corticosterone concentrations when compared to controls. Enzyme activity measurements are significantly lower in treated animals, demonstrating the efficacy of dsRNA treatment in inhibiting 11β HSD-1 activity.

### EXAMPLE 4

### Screens for dsRNA Fragments for Use in vivo

In order to develop an RNAi fragment for use in vivo, we first decided to measure the extent of the knockdown of several RNAi fragments in a cell culture screening system That meant that we needed to select cells that had ample native 11 b HSD-1 message. We have chosen to use pituitary cell lines (GH3 and GH4C1).

These cells were grown in Optimem medium for 48 h and then harvested, lysed and total RNA extracted using the Tri Reagent protocol. cDNA was synthesized and used as a template in a 30 cycle PCR reaction with the same 11β HSD 1 primers used to develop the template for 11β HSD-1 ds RNA.

Presented below is the gel electrophoresis of two pituitary cell lines (grown in duplicates) that were probed by PCR for 11β HSD-1 in duplicate.

[image]

By annealing a T7 promoter site on the 5′ end of a 500 bp 11β HSD-1 fragment we have developed a 500 bp dsRNA 11β HSD-1 fragment (using the Megascript kit by Ambion, Inc; Austin, Tex.).

Several μg's of the dsRNA fragment were then cut into 23 mer pieces using Invitrogen's Dicer kit. These diced fragments were then transfected into GH4C1 pituitary cells.

Cells were transfected using Invitrogen's Lipofectamine procedure, and were harvested 24 hours after application of 50 ng of diced 11β HSD-1 RNAi. Cells were lysed, cDNA was synthesized and then used as template for a 35 cycle multiplex PCR using primers for both β actin and 11β HSD-1. Presented below are the results of this experiment. 11β HSD-1 message was knocked down by 50% within 24 hours using as little as 50 ng of diced RNAi.

[image]

All of the above-noted published references are incorporated herein by reference. Numerous modifications and variations of the present invention are included in the above-identified specification and are expected to be obvious to one of skill in the art. Such modifications and alterations to the compositions and processes of the present invention are believed to be encompassed in the scope of the claims appended hereto.

1. A method for inhibiting the expression of a gene encoding 11β HSD-1 in a cell, the method comprising introducing into a cell an RNA comprising a double stranded structure (dsRNA) having a nucleotide sequence which is substantially identical to at least part of the gene encoding 11β HSD-1.

2. The method of claim 1, wherein the dsRNA is produced outside the cell.

3. The method of claim 2, wherein the dsRNA is produced recombinantly.

4. The method of claim 3, wherein the dsRNA is produced using a PCR-derived template.

5. The method of claim 1, wherein the dsRNA comprises a single self complementary RNA strand.

6. The method of claim 1, wherein the dsRNA comprises two separate complementary RNA strands.

7. The method of claim 1, wherein said dsRNA nucleotide sequence is substantially identical to SEQ ID NO: 1.

8. The method of claim 1, wherein said dsRNA nucleotide sequence is substantially identical to at least a portion of SEQ ID NO: 1.

9. The method of claim 1, wherein the dsRNA is substantially identical to the whole gene encoding 11β HSD-1.

10. An RNA comprising a double stranded structure (dsRNA) having a nucleotide sequence which is substantially identical to at least a part of the gene encoding 11β HSD-1.

11. The dsRNA of claim 10 having a nucleotide sequence which is substantially identical to SEQ ID NO: 1.

12. The dsRNA of claim 10 having a nucleotide sequence which is substantially identical to at least a portion of SEQ ID NO: 1.

13. The method of claim 1, wherein the dsRNA is produced by chemical synthetic means.

14. The method of claim 13, wherein the dsRNA is chemically modified.

15. The method of claim 13, wherein said dsRNA is between about 18 and 25 nucleotides in length.

16. A method for treating obesity in a patient comprising administering to said patient an RNA comprising a double stranded structure (dsRNA) having a nucleotide sequence which is substantially identical to at least part of the gene encoding 11β HSD-1.

17. The method of claim 15, wherein said dsRNA nucleotide sequence is substantially identical to SEQ ID NO: 1.

18. The method of claim 17, wherein the dsRNA nucleotide sequence is substantially identical to a portion of SEQ ID NO: 1.

19. The method of claim 17, wherein the dsRNA nucleotide sequence is produced recombinantly.

20. The method of claim 18, wherein the dsRNA nucleotide is produced synthetically and comprises between about 18 and 26 nucleotides.

21. The method of claim 20, wherein the dsRNA nucleotide is chemically modified.

22. The method of claim 20, wherein the dsRNA nucleotide is administered intravenously.

23. A pharmaceutical composition comprising a dsRNA having a nucleotide sequence which is substantially identical to at least part of the gene encoding 11β HSD-1 and a pharmaceutically acceptable carrier.

24. The pharmaceutical composition of claim 23, wherein said dsRNA nucleotide sequence is substantially identical to SEQ ID NO: 1.

25. The pharmaceutical composition of claim 24, wherein said dsRNA nucleotide sequence is substantially identical to a portion of SEQ ID NO: 1.

26. The pharmaceutical composition of claim 25, wherein the dsRNA nucleotide sequence is substantially identical to a portion of SEQ ID NO: 1.

27. The pharmaceutical composition of claim 24, wherein dsRNA nucleotide sequence is produced recombinantly.

28. The pharmaceutical composition of claim 26, wherein the dsRNA nucleotide is produced synthetically and comprises between about 18 and 26 nucleotides.

29. The pharmaceutical composition of claim 28, wherein said dsRNA nucleotide is chemically modified.

30. The pharmaceutical composition of claim 23 suitable for intravenous administration.

31. The pharmaceutical composition of claim 23 further comprising a vehicle that promotes the introduction of dsRNA into a cell.

32. A method of up regulating the activity of 11β HSD-1 in a mammal comprising administering to said mammal, a high fat diet for a period of time necessary to up regulate 11β-HSD-1 message and/or activity.

33. A method for treating a disease or disorder responsive to modulation of 11β HSD-1 gene expression, comprising administering to said patient an RNA comprising a double stranded structure (dsRNA) having a nucleotide sequence which is substantially identical to at least a portion of SEQ ID NO: 1.
